(19) United States
(12) Patent Application Publication
(10) Pub. No.: US 2025/0170053 A1
(43) Pub. Date: May 29, 2025

(54) LONG-LASTING RESORBABLE SUBCUTANEOUS IMPLANT WITH PROLONGED RELEASE OF PRE-CONCENTRATED PHARMACOLOGICALLY ACTIVE SUBSTANCE IN A POLYMER FOR THE ADJUVANT TREATMENT OF EPILEPSY, CHRONIC PAIN AND ANXIETY, AND METHOD

(71) Applicant: Edson Luiz Peracchi, Curitiba-PR (BR)

(72) Inventor: Edson Luiz Peracchi, Curitiba-PR (BR)

(21) Appl. No.: 18/868,715

(22) PCT Filed: Jun. 7, 2022
(86) PCT No.: PCT/BR2022/050204

§ 371 Date: Nov. 22, 2024

(30) Foreign Application Priority Data
May 27, 2022 (BR) 1020220104549

(51) Int. Cl. A61K 9/00; A61K 31/00; A61K 36/185

(52) U.S. Cl. CPC A61K 9/0024; A61K 31/658; A61K 36/3482

(57) ABSTRACT

Long-lasting biodegradable subcutaneous implant with prolonged release of pharmacologically active substance pre-concentrated in polymer for the treatment of epilepsy using from 10 to 1000 mg essential oil extracted from Cannabis sativa and/or Cannabis indica (full or broad spectrum), 10 to 500 mg natural or synthetic tetrahydrocannabidiol (THC) isolate, 10 to 500 mg natural or synthetic cannabidiol (CBD) isolate, proportional combination of broad spectrum essential oil and CBD isolate, proportional combination of full spectrum essential oil and natural or synthetic CBD isolate, or proportional combination of natural or synthetic THC isolate and CBD isolate in polymeric matrix for adjuvant treatment of epilepsy, chronic pain and anxiety.

### FIELD OF THE INVENTION

This invention comprises a long-lasting resorbable subcutaneous implant with prolonged release of a pharmacologically active substance pre-concentrated in a polymer for adjuvant treatment of epilepsy, chronic pain and anxiety.

### BASIC DESCRIPTION OF THE INVENTION

Epilepsy comprises a group of chronic neurological disorders in which recurrent seizures occur. These seizures result from excessive neuronal discharges and can take various forms.

Neuronal hyperexcitability can affect different parts of the brain. The region where the abnormal depolarization originates and the degree of propagation to other areas of the brain determine the type of seizure experienced by the patient. Thus, there are several types of seizures, which help in the clinical classification of epilepsy. The two main categories described are partial (focal) and generalized seizures. Each form is subdivided into simple (without consciousness loss) or complex (with consciousness loss).

In partial seizures, depolarization begins locally, in only one hemisphere of the brain, and there is usually no propagation. In this type of seizure, involuntary muscle contractions, anomalous sensory experiences, autonomic depolarization, or effects on mood and behavior may occur. In some individuals, partial seizures can turn into generalized seizures.

Generalized seizures involve the entire brain and are characterized by immediate loss of consciousness. This type of seizure can be subdivided into several categories (myoclonic, tonic, atonic, clonic), but the most important are tonic-clonic and absence seizures.

The development of epilepsy can result from brain injury (trauma or tumors, for example), or from other types of neurological diseases, including hereditary syndromes. About one third of cases are familial and involve genetic mutations. However, only 20% to 30% of individuals have a defined or suspected cause; for most patients there is no recognizable etiology.

Epilepsy can also be classified according to its etiology as idiopathic, characterized by the absence of structural lesion; symptomatic, in which there is proof of the lesion; or cryptogenic, where a lesion is not identified in imaging exams, but there are characteristics consistent with symptomatic epilepsy.

Epilepsy is considered a public health problem; it is one of the most common neurological disorders, with a wide distribution worldwide. According to data published by the World Health Organization (WHO), around fifty million people in the world are affected by the condition. Furthermore, around five million new diagnoses are made each year. In high-income countries, there are around forty-nine new diagnoses per year for every one hundred thousand people. In low-income countries, this number increases to one hundred and thirty-nine new cases per year for every one hundred thousand people.

This neurological disorder has significant economic implications. This is because the disease places a strain on health care systems, since, among other factors, individuals with epilepsy tend to have more physical problems (such as fractures and bruises from seizure-related injuries), as well as a higher rate of psychological disorders, including anxiety and depression. In the United States, for example, the cost associated with epilepsy is approximately $15.5 billion annually. In addition, absences from school and work, loss of productivity, and premature death further increase the economic burden. The risk of premature death among patients with epilepsy is up to three times greater than that of other patients.

Regarding diagnosis, patients with suspected epilepsy should undergo a complete medical history analysis, which should include information such as events that directly preceded the seizure, number of seizures in the last 24 hours, duration and description of the seizure, focal aspects, and duration of the postictal period. It seems relevant to remember that a single episode does not necessarily define the diagnosis of epilepsy; predisposing factors for recurrent seizures must also be considered.

Other important diagnostic tools include electroencephalography (EEG), neuroimaging, and laboratory tests. The need for laboratory tests is assessed according to the clinical context, and may include blood glucose, complete blood count, serum electrolyte levels (especially sodium), lumbar puncture in febrile patients, urine and toxicology tests.

It is estimated that up to 70% of individuals with epilepsy could live seizure-free if they were properly diagnosed and treated.

In most cases, treatment is carried out with medicines. Antiepileptic drugs (AEDs) aim to inhibit anomalous neuronal depolarization. Drugs can act through three main mechanisms of action, as follows: potentiation of the action of GABA (e.g., benzodiazepines); inhibition of sodium channel function (rufinamide); and inhibition of calcium channel function (valproate).

Commonly used antiepileptics include phenytoin, carbamazepine, valproate, ethosuximide, phenobarbital, diazepam, clonazepam, and clobazam. Some physicians choose to use newer drugs, such as vigabatrin, gabapentin, pregabalin, lamotrigine, felbamate, tiagabine, topiramate, levetiracetam, and oxcarbazepine.

Even with therapy using the AEDs, 10% to 30% patients are refractory, that is, they continue to have seizures at intervals of one month or less. These seizures can have a significant impact on the work and life of these individuals. In addition, the use of anticonvulsants is often limited by their adverse effects.

In recent years, several trials have investigated the efficacy of Cannabis sativa, Cannabis indica and their derivatives in controlling the symptoms of epilepsy.

One of these articles (“Cannabidiol in Patients with Treatment-Resistant Epilepsy: an Open-Label Interventional Trial”) evaluated the safety, tolerability, and efficacy of adjunctive therapy with cannabidiol (CBD) in the control of seizures. Patients aged one to thirty years with treatment-resistant epilepsy were included and received 2 to 50 mg/kg/day of oral CBD for twelve weeks. The treatment resulted in an average reduction of 36.5% in seizure frequency, and the safety and tolerability of the drug was acceptable. Only 3% of the individuals discontinued treatment due to some adverse event, which included drowsiness, diarrhea, fatigue, and decreased appetite as the most commonly reported events. It seems relevant to mention that the study included individuals with very poor prognosis, which highlights the potential for application of CBD, at least as adjunctive therapy.

The safety and efficacy of pure CBD therapies, as well as CBD-enriched C. sativa extracts, were also evaluated in a meta-analysis entitled “Potential Clinical Benefits of CBD-Rich Cannabis Extracts over Purified CBD in Treatment-Resistant Epilepsy: Observational Data Meta-Analysis”. The article compiled the results of eleven observational studies, totaling six hundred and seventy patients treated for refractory epilepsy. The doses used ranged from 2 to 50 mg/kg/day of pure CBD or oral CBD-enriched extract, and the intervention period was from three months to one year, depending on the study. As a result, two-thirds of the patients (64%) reported an improvement in seizure frequency, and 39% reported a decrease of at least 50% in this frequency. Furthermore, in addition to this direct therapeutic effect, patients also reported improvements in secondary aspects, such as: improved perception (52% of patients), sleep quality (31%), mood (30%), behavior (20%), cognition (7%) and motor skills (7%). These results should also be considered, since they revealed an improvement in the quality of life of patients with epilepsy and their families.

Products based on C. sativa containing tetrahydrocannabidiol (THC) may also improve epileptic patients. The article “A Prospective Open-Label Trial of a CBD/THC Cannabis Oil in Dravet Syndrome” evaluated the oral use of a combination of CBD and THC in a 50:1 ratio (100 mg CBD/mL; 2 mg THC/mL) in patients with treatment-resistant epilepsy. The objective was to evaluate the dose, tolerability, and efficacy of the combination as adjuvant treatment in children with Dravet syndrome. A total of twenty children received doses between 2 and 16 mg/kg/day of CBD, and 0.04 and 0.32 mg/kg/day of THC for 20 weeks. The frequency of motor seizures was reduced by an average of 70.6%, and 63% patients showed a reduction of at least 50% in the number of seizures. Furthermore, there was a statistically significant improvement in quality of life, reduction in spike activity observed in the EEG and, according to those authors, the use of the combination was well tolerated by the patients.

One point to consider is that oral administration of CBD may have some limitations, including gastrointestinal adverse effects such as nausea, vomiting, and diarrhea, as well as low bioavailability. Furthermore, the oral form generates inconsistent plasma levels (which vary widely between patients), and significant first-pass metabolism.

Pain has a vital protective function, to alert and protect us. It becomes a problem when it loses this functionality and becomes a nuisance. It can be classified according to its origin or duration. Acute pain is short-lived and serves to alert us to possible damage to the body. This painful sensation goes away after the stimulus ceases, or when resulting from a pathology, after it has been cured. Chronic pain, as its name suggests, is long-lasting and is considered for academic and scientific purposes as pain that persists for more than six months. This kind of pain loses its protective and alert function and becomes a pathology.

Although acute pain can be considered adaptive, in some situations it evolves into a chronic state, becoming a health problem, initially on a personal scale, later on a public scale. Because it generates a certain degree of physical and functional disability, whether temporary or permanent, the condition can result in high costs to health systems, with a major impact on patients' quality of life. Other factors such as social isolation, changes in libido and feelings of hopelessness lead to other comorbidities such as anxiety, depression, and insomnia, among others, including obesity. Chronic pain is a pathological condition of enormous clinical, social and economic significance. Thus, in 2019, the IASP (International Association for the Study of Pain) didactically identified the clinical conditions associated with chronic pain for a better scientific and therapeutic approach.

However, despite chronic pain showing high prevalence and being a disabling pathology, current treatments have not been able to treat satisfactorily all patients, in addition to presenting considerable adverse effects, revealing the need for new and more efficient painkillers.

Cox 1 and 2 inhibitors are already available in the market. These drugs prevent the transformation of arachidonic acid into inflammatory mediators, opioid drugs, non-steroidal anti-inflammatory drugs, antidepressant drugs, anticonvulsant drugs and local anesthetics, all of which are widely used in the treatment of pain.

In the last few decades, a new target to relieve pain was discovered from the modulation of an endogenous system and by using an old medication extracted from plants in preparations with Cannabis sativa and Cannabis indica, which have been used as painkillers for centuries.

Studies developed by Mechoulam, Devane and Munro revealed the existence of a functional endocannabinoid endogenous system (ECS). Such findings were fundamental to elucidate the mechanisms and sites of action of the phytocannabinoids in pain modulation.

The endocannabinoid system is widely distributed throughout the body, where CB2 metabotropic receptors are more associated with the immune system cells and CB1 receptors are associated with the central nervous system (CNS). The activation of such receptors results in the inhibition of the release of neurotransmitters in a series of intracellular cascades.

In the CNS at a supraspinal level, CB1 receptors are highly expressed in the periaqueductal gray matter (PAG) and in the nuclei of the ventromedial dorsal medulla (RVM), and their activation reduces the sensation of pain and in the nerve terminals, CB1 activation inhibits the sensitizing effects of the receptors, thus inhibiting the feeling of pain.

Pain is the main medical indication for the use of Cannabis sativa and Cannabis indica at the treatment level, representing around 70% of all patients with prescription for the medicinal use of such drugs. The plant use as painkiller was reported by the Chinese in the oldest pharmacopeia in the world, the Pents'ao Ching, which was compiled in the first century of this era based on oral traditions transmitted since the time of Emperor Shen-Nung, who lived during the years 2,700 BC.

Cannabidiol is the best-known non-psychoactive (non-psychotomimetic) phytocannabinoid, mainly due to its attribution to the medicinal effects of Cannabis sativa and Cannabis indica. The analgesic effects of CBD have already been demonstrated experimentally by Maayah ZH, Takahara S, Ferdaoussi M, Dyck JRB., in the article published in 2020 with the title The molecular mechanisms that underpin the biological benefits of full-spectrum cannabis extract in the treatment of neuropathic pain and inflammation and may be associated with the suppression of receptors such as TRPV1, GPR55, NMDA and α1-adrenergic receptors, T-type calcium channels and μ-/δ-opioid receptors.

Research results suggest that CBD isolates require higher doses and are more likely to cause side effects than holistic whole-plant formulas. This is due to the so-called entourage effect, a synergistic relationship between the active compounds found in the plant. Thus, the administration of oils known as FULL or BROAD SPECTRUM, which contain other cannabinoids, terpenes and flavonoids in addition to CBD, are more comfortable and economical.

Anxiety and fear are essential emotional responses for our survival, which occur in anticipation of potential danger or threats. When expressed appropriately, they can save lives, but when they become excessive, they interfere with individuals' behavior and are then considered psychiatric disorders.

Anxiety and related disorders such as phobias, panic, obsessive-compulsive disorder and post-traumatic stress are the most common psychiatric illnesses, which cause social and economic losses for the individual and for society. These disorders are associated with disturbed cognition and emotional regulation, with psychological symptoms such as excessive fear, apprehension, problems with concentration and sleep, and somatic symptoms such as tachycardia, palpitations, sweating and obesity.

The treatment usually includes a combination of psychological therapy and the administration of drugs, which are many times limited thus presenting temporary efficacy and countless undesirable side effects such as anxionegenesis, insomnia, restlessness, headaches, gastrointestinal and appetite disorders, and even sexual dysfunction, which reinforces the need for new therapeutic options for the treatment of these pathologies.

New knowledge about the brain regions and neuronal circuits that regulate anxiety has been obtained in the last few years and several pharmacological and genetic studies support the role of the endocannabinoid system (ECS) as an important regulator of such behavior.

Although there is no consensus among the medicine councils on the use of cannabidiol (CBD) in the treatment of psychiatric disorders such as anxiety, several studies have reported its antianxiety, antidepressant, antipsychotic, anti-inflammatory, and neuroprotective properties and the body of evidence regarding this theme has grown significantly.

The isolation of phytocannabinoids led to the identification of biological targets through which they provide their effects, including type 1 (CB1) and type 2 (CB2) cannabinoid receptors. Next, the discovery of endogenous ligands for these receptors occurred. These are lipid messengers called endocannabinoids, such as anandamide (AEA) and 2-arachidonoylglycerol (2-AG).

Endocannabinoids, the enzymatic machinery responsible for their synthesis and degradation, and the aforementioned receptors constitute an important system called endocannabinoid, which, although not yet fully understood, is considered a fundamental regulatory apparatus related to almost all physiological aspects of mammals, presenting important neuromodulator activity and in the maintenance of homeostasis.

The endocannabinoid system (ECS) appeared as a central integrator linking the perception of external and internal stimuli to neurophysiological and behavioral results (such as the reaction to fear, anxiety, and stress control), thus allowing the body to adapt to the changes in the environment. The ECS signaling seems to control the stimuli that evoke fear and to adjust proper behavioral responses, which are essential for the long-term viability of the body, homeostasis and stress resilience. The deregulation of this system might result in psychiatric disorders and obesity.

The literature reporting studies with rodents by Guimarães FS, Chiaretti TM, Graeff FG, Zuardi AW in the article Antianxiety effect of cannabidiol in the elevated plus-maze, of 1990, showed that the systemic CBD administration produced antianxiety effects in the elevated plus-maze test along with other tests related to compulsive disorders such as the OCD and PTSD, respectively. In 2012, Hsiao YT, Yi PL, Li CL, Chang FC in the article Effect of cannabidiol on sleep disruption induced by the repeated combination tests consisting of open field and elevated plus-maze in rats, published in the Neuropharmacology journal, reported the importance of the CBD use in the treatment of obsessive-compulsive disorders called OCD.

The need to find new ways of managing anxiety disorders is clear since they have become more common thus affecting a significant part of the population.

The treatments available are precarious and usually involve a combination of drugs, including benzodiazepines and antidepressants. Those medicines show disadvantages such as the risk of dependence and withdrawal effect syndromes, as well as side effects such as cognitive and psychomotor impairment, delayed action and the need for a thorough dosage control. The CBD properties that reduce inflammation and oxidative stress associated with neurotoxicity allied to the absence of psychoactive effects and potential abuse, in addition to having weak sedative properties compared to benzodiazepines make CBD an excellent candidate for a new approach to the treatment of psychiatric disorders, especially anxiety-related disorders.

Clinical studies in healthy volunteers and in patients with anxiety disorders confirm preclinical findings that cannabidiol has significant anxiolytic activity, with an inverted U-shaped dose-response curve in humans.

Another important fact is that chronic diseases, such as epilepsy, chronic pain and anxiety, require the patient to take at least one medication, often more than once a day, for an extended period in order to treat the disease. It is now known that one of the factors leading to poor adherence to drug treatment for epilepsy is the number of times an antiepileptic drug is taken per day, anxiety and chronic pain.

Increased number of medications and number of doses per day tend to reduce adhesion. This is the main disadvantage reported in articles investigating these treatments in clinical practice.

According to the World Health Organization, therapeutic adherence is determined by the interaction between the health system and its team, socioeconomic factors, factors related to the patient, treatment and disease. Adhesion to treatment is one of the main factors related to the success or failure of a drug therapeutic approach. Many times, the therapeutic result is not as positive as expected due to the patients' conduct, for several reasons that prevent them from following the treatment correctly and thus not obtaining the expected effect.

In general, the increase in the number of pills taken by the patient in one day reduces the treatment by around 20%, and the medication used in multiple doses also reduce adhesion when compared to a single-dose treatment.

Regarding epilepsy, therapeutic adhesion reduces with the increased frequency of drug administration: the posology of once a day shows an adhesion degree of around 87%; twice a day has an 81% adhesion degree, falling to 77% when the medication must be taken three times a day, and 39% for drugs taken four times a day.

However, to keep the patient crisis-free, it is necessary to use appropriate drugs and to continue the dosage regimen in a disciplined manner. Therefore, the importance of lack of adhesion as one of the critical factors of therapeutic failure in epilepsy, chronic pain and anxiety treatments becomes evident.

The oral route traditionally used in the treatment of epilepsy with Cannabis sativa and Cannabis indica derivates shows the disadvantage of low therapeutic adhesion. The most efficient way to increase adhesion to the treatment by the patients is to simplify the medication dosage. In many chronic diseases, such as epilepsy, chronic pain, and anxiety, the development of prolonged-release medication has simplified the dosage.

Another disadvantage of the oral route for epilepsy, chronic pain and anxiety medication is in the ease of misusing the prescribed medication, increasing the risk of sub or supra physiological doses, making patients more susceptible to the adverse effects of the medication or even suffering from the adverse effects of the lack of medication, thus compromising their general condition during treatment.

However, it is possible to find another option known as bioabsorbable implants or pellets of essential oil extracted from Cannabis sativa and Cannabis indica (full or broad spectrum), tetrahydrocannabidiol (THC) isolate, cannabidiol (CBD) isolate, combination of broad spectrum essential oil and CBD isolate or synthetic, combination of full spectrum essential oil and CBD isolate or synthetic and/or THC isolate, and combination of synthetic THC and and/or synthetic CBD isolate in preparations with variable doses adapted to each pathology. Such implants guarantee sustained release of the drug for a long period of time to treat the disease, making the treatment independent of the patients' initiative to take the medication and improve their clinical symptoms.

The term “full spectrum” refers to the essential oil extracted from the Cannabis sativa and the Cannabis indica in full. The term “broad spectrum” refers to the essential oil extracted from Cannabis sativa and Cannabis indica with all cannabinoids and other compounds, except for the THC.

The terms “implant” or “pellet” refer to the pharmaceutical form already consolidated in the official collections of norms for medication and pharmaceutical substances. They are characterized as solid and sterile preparations of suitable size and format for parenteral implant and release of active substances throughout an extended period.

The terms “prolonged release”, “slow release” or “sustained release” refer to the way the medication is released by the implant, which occurs continuously and gradually over time rather than an immediate and concentrated release of the drug in the body.

Biodegradable polymers or bioerodible polymers refer to a polymer that degrades in vivo and whose erosion over time occurs concomitantly with and/or after the release of the therapeutic agent. A biodegradable polymer may be a homopolymer, copolymer, or a polymer comprising more than two polymer units. In some cases, a biodegradable polymer may include a blend of two or more homopolymers or copolymers.

Biodegradable implants or bioerodible implants might be understood as implants that present some mechanism that promote the gradual reduction of their mass over the time of release. The forces involved in this mass reduction can result from cellular interaction or shear forces on the implant surface.

### DESCRIPTION OF THE STATE OF THE ART

As regards patent registrations related to resorptive implants, paper U.S. Pat. No. 4,957,119 (Contraceptive implant) mentions an implant of polymeric material that can release a contraceptive agent for a relatively long time when adjusted subcutaneously or locally. The implant comprises an ethylene/vinyl acetate copolymer core material that acts as a matrix for a contraceptive substance, an ethylene/vinyl acetate membrane encasing the core material and a contact layer at the interface of the core material and membrane which prevents the separation of the core material from the membrane.

Despite the application for a resorptive implant, registration U.S. Pat. No. 4,957,119 uses different active substances, its production is carried out by means of extrusion and the release period of the active substance is very long (at least 1year), as distinguished from the resorbable subcutaneous implant of this invention.

The second register number U.S. Pat. No. 9,980,850 (Bioerodible contraceptive implant and methods of use thereof) describes a bio erodible contraceptive implant and methods of its use in the form of a controlled-release bio erodible granule for subdermal implantation. The bio erodible sediment supplies the sustained release of a contraceptive agent for a prolonged time. Bioerosion products are soluble in water, bio resorbed, or both, thus preventing the need for surgical removal of the implant.

In the same way as the first previously cited records, this record U.S. Pat. No. 9,980,850 similarly uses different active substances, the release period of the active substance is very long (from 6 months to 4 years) and the preferred method for manufacturing them is the hot fusion molding process.

When observing the existing deficiencies and problems in conventional epilepsy, chronic pain, and anxiety treatments, this invention aims to become a tool helping those patients to control epilepsy and to improve their clinical condition.

The treatment proposed does not depend on the patients' memory or commitment to the correct use of the medication, and by means of prolonged release and lower dosage of the active substance, it can reduce adverse symptoms observed in the oral intake of the drug as well as prevent its hepatic metabolization since the implant releases it directly in the blood stream. In addition, at the end of the implant duration, it is not necessary to remove it, only the reinsertion of new implants is required to keep the treatment.

### DESCRIPTION OF THE DRAWINGS

The following drawings are attached to provide a better understanding of this invention:

FIG. 1—shows a representation of the chemical structure of the cannabidiol (CBD) and tetrahydrocannabidiol (THC) substances;

FIG. 2—shows a dimensional project of the bioabsorbable implant and active substance;

FIG. 3—shows a dimensional project of the non-absorbable implant and active substance.

### DETAILED DESCRIPTION OF THE INVENTION

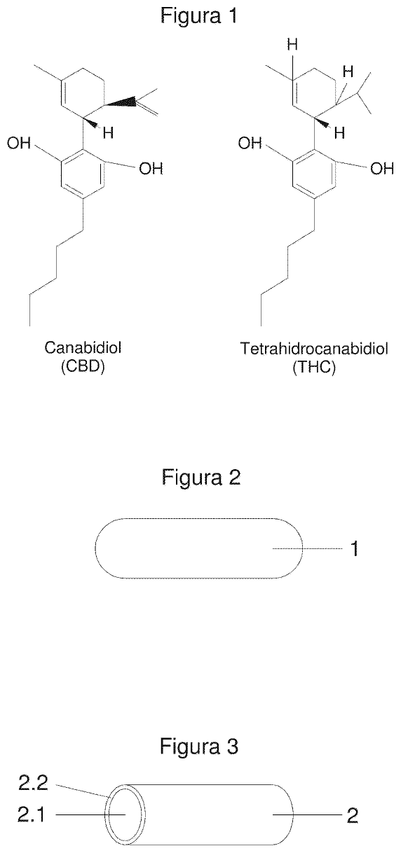

This invention is a biodegradable implant with dimensional design of the bioabsorbable implant with essential oil extracted from Cannabis sativa and Cannabis indica (full or broad spectrum), tetrahydrocannabidiol (THC) isolate, cannabidiol (CBD) isolate, combination of broad spectrum essential oil and or synthetic CBD isolate, combination of full spectrum essential oil and or synthetic CBD isolate, and association of THC CBD isolate and or synthetic for adjuvant treatment of epilepsy, chronic pain, and anxiety in polymeric matrix. The implant is inserted subcutaneously and guarantees continuous release of the active substance for a prolonged period. This release ensures an efficient, steady and prolonged serum level of the drug for the treatment of epilepsy, chronic pain and anxiety. “Active substance”, “active” or “drug” refers to a medication for the adjuvant treatment of epilepsy, chronic pain and anxiety which may include: essential oil extracted from Cannabis sativa and Cannabis indica (full or broad spectrum), tetrahydrocannabidiol (THC) isolate, cannabidiol (CBD) isolate, association of broad spectrum essential oil with CBD isolate or synthetic, association of full spectrum essential oil with CBD isolate or synthetic, and association of THC with CBD isolate and/or synthetic. The chemical structures of the isolated substances are shown in FIG. 1

The implant of this invention may contain the agent for the treatment of epilepsy, chronic pain and anxiety, but it is preferably composed of particles of the active extracted from the oil of Cannabis sativa and Cannabis indica (full or broad spectrum), tetrahydrocannabidiol (THC) isolate, cannabidiol (CBD) isolate, associated broad spectrum essential oil and CBD isolate or synthetic, combination of full spectrum essential oil and CBD isolate or synthetic, and combination of THC and CBD isolate and/or synthetic, homogeneously dispersed in a bioerodible and bioabsorbable polymeric matrix. This polymeric matrix can be formed by a polymer or a mixture of polymers. The amount of active substance present in the implant may range from 10 to 1000 mg essential oil extracted from Cannabis sativa and/or Cannabis indica (full or broad spectrum), 10 to 500 mg natural or synthetic tetrahydrocannabidiol (THC) isolate, 10 to 500 mg natural or synthetic cannabidiol (CBD) isolate, proportional association of broad spectrum essential oil and natural or synthetic CBD isolate, proportional association of full spectrum essential oil and natural or synthetic CBD isolate, or proportional association of THC isolate and natural or synthetic CBD isolate: its composition might contain from 1% to 20% biodegradable polymer in proportion to its weight.

The biodegradable polymer used can be Poly (D-lactic acid), Poly (L-lacticacid), Poly (racemic lactic acid), Poly (glycolic acid), Poly (caprolactone), methylcellulose, ethylcellulose, hydroxypropylcellulose (HPC), hydroxypropylmethylcellulose (HPMC), polyvinylpyrrolidone (PVP), poly (vinyl alcohol) (PVA), poly (ethylene oxide) (PEO), polyethylene glycol, starch, natural and synthetic rubber and wax.

Implants can have any size, shape or structure that make their manufacture and subcutaneous insertion easier. However, to achieve a more consistent and uniform release of the active substance it is necessary to use geometric shapes that maintain its surface area over time.

Therefore, the implant developed and demonstrated in this application adopts a cylindrical shape (1), like a rod with straight or rounded tips, between 2 to 30 mm long and a diameter of 1 to 10 mm. A schematic drawing of an example of the implant dimensions (1) is found in FIG. 2.

The implant manufacture with essential oil extracted from Cannabis sativa and Cannabis indica (full or broad spectrum), tetrahydrocannabidiol (THC) isolate, cannabidiol (CBD) isolate, combination of broad spectrum essential oil and CBD isolate or synthetic, combination of full spectrum essential oil and CBD isolate or synthetic, and THC and CBD isolate and/or synthetic combination for adjuvant treatment of epilepsy, chronic pain and anxiety can be carried out from the addition of 10 to 1000 mg essential oil extracted from Cannabis sativa and Cannabis indica (full or broad spectrum), 10 to 500 mg natural or synthetic tetrahydrocannabidiol (THC) isolate, 10 to 500 mg natural or synthetic cannabidiol (CBD) isolate, proportional association of broad spectrum essential oil and natural or synthetic CBD isolate, proportional association of full spectrum essential oil and natural or synthetic CBD isolate, or proportional association of natural or synthetic THC isolate and CBD isolate in the biodegradable polymeric matrix chosen in the 1% to 20% proportion in relation to the drug weight with the formation of a homogeneous mixture. If the polymer solvent is not the same as the drug solvent, it will remain dispersed as particles or suspension and a mixer can be used to make the solution homogeneous. This solution is then dried and shaped to form the implant (1) or any other desired format.

Another possible way to manufacture the implant with essential oil extracted from Cannabis sativa and Cannabis indica (full or broad spectrum), tetrahydrocannabidiol (THC) isolate, cannabidiol (CBD) isolate, association of broad spectrum essential oil and natural or synthetic CBD isolate, association of full spectrum essential oil and natural or synthetic CBD isolate, and association of natural and/or synthetic THC and CBD, for adjuvant treatment of epilepsy, chronic pain and anxiety is from the mixture of 10 to 1000 mg essential oil extracted from Cannabis sativa and Cannabis indica (full or broad spectrum), 10 to 500 mg natural or synthetic tetrahydrocannabidiol (THC) isolate, 10 to 500 mg natural or synthetic cannabidiol (CBD) isolate, proportional association of broad spectrum essential oil and natural or synthetic CBD isolate, proportional association of full spectrum essential oil and CBD isolate, or proportional association of natural or synthetic THC isolate and CBD isolate, in each implant and from 1% to 20% of the biodegradable polymeric matrix chosen in relation to the drug weight in its dried powdered form. The drug and the polymeric matrix are added into a proper container and the mixture is then homogenized.

The mixture of active substances to manufacture the implant can be shaped by means of pressure or heat, so that neither the drug efficacy is compromised, nor the polymeric material is degraded. The technical options to shape the implant include injection, heat, compression or extrusion shaping.

To prepare this invention, the technique chosen was compression. In this technique, the mixture of powder actives was placed in a mold and mechanical force was applied to the mixture generating compression of particles and consequent shaping of the implant (1). Next, the implant was filled and sterilized with the agent for the adjuvant treatment of epilepsy, chronic pain and anxiety. Its sterilization can be obtained using heat (approximately 90° C.), e-beam, or gamma rays.

The implant might present a coating polymeric membrane between 0.1 and 0.7 mm thick. The polymer used to coat must be bioabsorbable and enable the active substance passage. The implant coating is preferably applied by dipping the implant in a polymeric solution. The coating might cover the implant's whole surface including the edges, or only its longitudinal surface leaving the edges uncovered, or even only the implant edges without covering its length. Polymers that can be used to coat the implant include poly (glycolic lactic-co-acid acid) (PLGA) and D,L-lactic acid copolymers.

Also, another option of implants to treat epilepsy, chronic pain and anxiety is the use of non-biodegradable implants. Non-biodegradable or non-bio erodible implants (2) (FIG. 3) present a core (2.1) formed by polymeric matrix in the percentage from 1% to 20% in relation to the drug weight. In this case, it contains from 10 to 1000 mg essential oil extracted from Cannabis sativa and Cannabis indica (full or broad spectrum), 10 to 500 mg natural or synthetic tetrahydrocannabidiol (THC) isolate, 10 to 500 mg natural or synthetic cannabidiol (CBD) isolate, proportional combination of broad spectrum essential oil and natural or synthetic CBD isolate, proportional combination of full spectrum essential oil and natural or synthetic CBD isolate, or proportional combination of natural and/or synthetic THC isolate and CBD isolate with the core covered by a non-degradable polymeric membrane (2.2), which controls the drug release rate.

The material used to obtain the polymeric membrane that coats the implant might be silicone, urethane, acrylates and its copolymers, polyvinylidene fluoride copolymers, polyethylene vinyl acetate-ethylene vinyl, dimethylpolysiloxane. This membrane thickness may range between 0.2 and 1 mm and is shaped using specific equipment. After shaping the membrane from the polymeric material, the active mixture is inserted, forming the implant core (2.1) (2). The polymers used in the polymeric matrix and the mixture adopt the same compounds and process as that of the bioabsorbable implant.

Drug release in this system occurs via diffusion, at a relatively constant rate and it is possible to alter the speed of the drug release by changing the membrane thickness or material. This system does not require the removal of the implant at the end of the treatment.

The technological innovation presented provides different possibilities for the adjuvant treatment of epilepsy, chronic pain and anxiety as defined by the medical criteria: (a) therapy with broad spectrum essential oils extracted from Cannabis sativa and/or Cannabis indica; (b) therapy with full spectrum essential oil extracted from Cannabis sativa and/or Cannabis indica; (c) therapy with CBD isolate and/or synthetic implants; (d) therapy with THC isolate and/or synthetic implants; (e) therapy combining one or more of the substances listed (a, b, c, d) in the same implant; (f) therapy combining one or more of the substances listed (a, b, c, d) in distinct implants.

Regardless of the therapeutic scheme chosen, to define individualized treatment to each patient, professionals need to take into account the classification of epilepsy, chronic pain and anxiety, the evaluation of the clinical condition, and the results of the relevant exams. This information should define the concentration patterns and ideal approach for everyone.

During the treatment, the dose might be adjusted by inserting additional implants if needed. Furthermore, in the event of rejection or any adverse reaction after the implant insertion, it can be removed within the first couple of days of treatment.

The adjuvant treatment of epilepsy, chronic pain and anxiety with such implants has the advantage of providing from 8% to 20% of the oral doses. The dose reduction occurs due to the implementation of the drug in the subcutaneous layer, thus preventing its first-passage metabolism, and absorption by the gastrointestinal tract occurring throughout one's life. Thus, it is possible to reduce the necessary dose to keep the active bioavailability. The active substance prolonged release via the implant prevents plasmatic sub or supra-physiological concentrations, that is, the “peaks and troughs” that occur in the oral route. Moreover, the implant duration is approximately three, six, or twelve months, which are the periods proposed between the implant insertions.

For treatments with full spectrum or broad-spectrum essential oil extracted from Cannabis sativa and/or Cannabis indica, the dosage suggested might vary from 15 mg/day to 3000 mg/day. In a three-month (90 days) treatment with such dosage, the patient uses from 1.4 g to 270 g, while using the implant the dosage decreases to around 0.11 g (8% of 1.4 g) to 54 g (20% 270 g) in the same time interval, reaching a similar therapeutic effect and providing the same benefits listed above.

For treatments with CBD isolate, the suggested dosage might vary from 45 mg/day to 225 mg/day. In a three-month treatment (90 days) with such dosage, the patient would use from 4.1 g to 20.3 g medicine. With the implant, the dosage could be decreased from around 0.33 g (8% of 4.1 g) to 4.1 g (20% of 20.3 g) in the same time interval, achieving a similar therapeutic effect in addition to all benefits listed above.

For treatments with THC isolate, the suggested dosage might vary from 5 mg/day to 25 mg/day. In a three-month treatment (90 days) with such dosage, the patient would use from 0.5 g to 2.3 g. With the implant, the dosage could be decreased from around 0.04 g (8% of 0.5 g) to 0.5 g (20% of 2.3 g) in the same time interval, achieving a similar therapeutic effect in addition to all benefits listed above.

The implant use proposed is safe and efficient in the treatment of epilepsy, chronic pain and anxiety since the therapeutic approach does not depend on the patients' willingness or discipline to guarantee the medication action, thus ensuring the use of the prescribed dose and a regular treatment. The use of such implants in the therapeutic approach prevents discontinuity without medical assistance and guarantees suitable treatment and its efficacy. In addition, the invention prevents the patient from misusing the medication by taking more medicine than the dose recommended by the doctor protecting them from becoming more susceptible to undesirable side effects and worsening their clinical situation.

The implant with essential oil extracted from Cannabis sativa and/or Cannabis indica (full or broad spectrum), tetrahydrocannabidiol (THC) isolate, cannabidiol (CBD) isolate, association of broad spectrum essential oil and CBD isolate or synthetic, association of full spectrum essential oil and CBD isolate or synthetic, and association of THC isolate and CBD isolate and or synthetic for the adjuvant treatment of epilepsy, chronic pain and anxiety prevents the “peaks and troughs” of the oral route. The implant action mechanism in the body enables more continuous release of the active substance for an extended period. The daily release of sufficient amounts of the drug keeps the medication serum levels efficient, thus improving the patients' quality of life and treatment maintenance rates.

Another advantage of the implants described here is the release of medication in the blood stream, which limits side effects, makes its action more efficient and prevents the drug first-passage metabolization. Dosage simplification and decreased frequency of intake promotes greater adherence to the treatment.

1. A long-lasting resorbable subcutaneous implant having a prolonged release of a pharmacologically active substance for the treatment of epilepsy, chronic pain and anxiety, said implant containing;
a biodegradable and bioabsorbable polymeric matrix infused with said pharmacologically active substance, wherein said pharmacologically active substance includes:
10 mg to 1000 mg of an essential selected from a group comprising full spectrum Cannabis sativa, broad spectrum Cannabis sativa, full spectrum Cannabis indicia and broad spectrum Cannabis indica,
10 mg to 500 mg of an isolate selected from a group consisting of natural tetrahydrocannabidiol isolate, synthetic tetrahydrocannabidiol isolate, natural cannabidiol isolate and synthetic cannabidiol isolate, proportional combination of broad spectrum essential oil and natural or synthetic CBD isolate, proportional combination of full spectrum essential oil and natural or synthetic CBD isolate, or proportional combination of THC isolate and natural or synthetic CBD isolate in the form of particles
wherein said pharmacologically active substance is dispersed homogeneously in said biodegradable and bioabsorbable polymeric matrix, and wherein said polymeric matrix contains from 1% to 20% by weight of a biodegradable polymer.

2. The implant according to claim 1, wherein said biodegradable polymer is selected from a group consisting of Poly (D-lactic acid), Poly (L-lactic acid), Poly (racemic lactic acid), Poly (glycolic acid), Poly (caprolactone), methylcellulose, ethylcellulose, hydroxypropylcellulose (HPC), hydroxypropylmethylcellulose (HPMC), polyvinylpyrrolidone (PVP), poly (vinyl alcohol) (PVA), poly (ethylene oxide) (PEO), polyethylene glycol, starch, natural and synthetic rubber and wax.

3. The implant, according to claim 1, wherein said implant has a cylindrical shape, in the form of a rod, provided with straight or rounded tips between 2 mm and 30 mm long and with a diameter of 1 mm to 10 mm.

4. The implant, according to claim 1, further including a polymeric membrane coating of at least part of the implant, with a thickness between 0.1 mm to 0.7 mm, wherein said coating is selected from a group consisting of poly (lactic-co-glycolic acid) and D, L-lactic acid copolymers.

5. The implant, according to claim 1 further including a non-biodegradable core formed by said polymeric matrix wherein said core is covered by a non-degradable polymeric membrane.

6. The implant according to claim 4, wherein said polymeric membrane is made from a material selected from a group consisting of silicone, urethane, acrylates and its copolymers, polyvinylidene fluoride copolymers, polyethylene vinyl acetate-ethylene vinyl, dimethylpolysiloxane,.

7. A method comprising:
mixing active ingredients, wherein the active ingredients include an essential oil and an isolate,
wherein said essential oil is 10 mg to 1000 mg of an oil extracted from a group consisting of Cannabis sativa Cannabis indica,
wherein said isolate is 10 mg to 500 mg of an isolate selected from a group consisting of tetrahydrocannabidiol isolate, and cannabidiol isolate
infusing said active ingredients into a biodegradable polymeric matrix, which is then dried and shaped as an implant.

8. The method according to claims 7, said active ingredients are in a dried powder when mixed with said polymeric matrix to produce a homogenized mixture.

9. The method according to claim 8, wherein said homogenized mixture is subjected to compression shaping where mechanical force is applied to said homogenized mixture with consequent particle compression and implant shaping followed by sterilization.
